(19) United States
(12) Patent Application Publication
(10) Pub. No.: US 2024/0125393 A1
(43) Pub. Date: Apr. 18, 2024

(54) VALVE AND VALVE COMPONENTS FOR AN ENDOSCOPE

(71) Applicant: Boston Scientific Scimed, Inc., Maple Grove, MN (US)

(72) Inventor: Nathan Thomas Cummings, Worcester, MA (US)

(73) Assignee: Boston Scientific Scimed, Inc., Maple Grove, MN (US)

(21) Appl. No.: 18/485,771

(22) Filed: Oct. 12, 2023

Related U.S. Application Data
(60) Provisional application No. 63/415,711, filed on Oct. 13, 2022.

(51) Int. Cl. F16K 3/24; A61B 1/015

(52) U.S. Cl. CPC F16K 3/243; A61B 1/015

(57) ABSTRACT

Devices, systems, and methods for a valve for a medical device. The valve may include a valve stem. An elongate body of the valve stem may include a first opening and a second opening fluidly coupled to one another via a lumen. The valve stem may include a first seal and a second seal extending circumferentially around the elongate body. The elongate body, the first seal, and the second seal may be formed from a single material. The single material may be a thermoplastic elastomer or other suitable material. The valve stem may include recessed portion proximate the seals of the valve stem to facilitate bending or flexing of the seals as the valve stem translates within a valve well.

### CROSS-REFERENCE TO RELATED APPLICATIONS

This application claims the benefit of U.S. Provisional Patent Application Ser. No. 63/415,711 filed on Oct. 13, 2022, the disclosure of which is incorporated herein by reference.

### TECHNICAL FIELD

This disclosure relates generally to valve assemblies and methods, and particularly to valve stems, seals, and methods for an endoscope.

### BACKGROUND

A wide variety of intracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems. In a first example, a valve stem for a medical device may comprise an elongate body, a first opening in the elongate body, a second opening in the elongate body, a lumen extending from the first opening to the second opening, a first seal extending circumferentially around the elongate body at a location proximal of the first opening, a second seal extending circumferentially around the elongate body at a location distal of the first opening, and wherein the elongate body, the first seal, and the second seal may be formed from a single material.

Alternatively or additionally to any of the examples above, the elongate body, the first seal, and the second seal may be monolithic.

Alternatively or additionally to any of the examples above, the valve stem may further include a third seal extending circumferentially around the elongate body at a location distal of the second seal, and the third seal may be formed from the single material used to form the elongate body, the first seal, and the second seal.

Alternatively or additionally to any of the examples above, the valve stem may further comprise a first recess extending circumferentially around the elongate body at a first axial location, and a second recess extending circumferentially around the elongate body at a second axial location spaced from the first axial location, and the first seal may extend from the first recess to the second recess.

Alternatively or additionally to any of the examples above, the single material may be a thermoplastic elastomer.

Alternatively or additionally to any of the examples above, a wall thickness of the elongate body between the lumen and an outer surface of the elongate body at an axial location between the first seal and the second seal may be in a range of 1.00 millimeters (mm) and 2.00 mm.

Alternatively or additionally to any of the examples above, a wall thickness of the elongate body between the lumen and an outer surface of the elongate body at an axial location between the first seal and the second seal may be at least 1.50 mm.

Alternatively or additionally to any of the examples above, the single material may have a durometer in a range of 50 Shore A to 75 Shore A.

Alternatively or additionally to any of the examples above, the first opening is a radial opening and the second opening is an axial opening in a distal end of the elongate body.

Alternatively or additionally to any of the examples above, an engagement portion may extend proximally from the elongate body.

In another example, a valve stem for a medical device, where the valve stem is configured to translate within a valve well of the medical device, may include an elongate body formed from a single material, a first opening in the elongate body, a second opening in the elongate body, a lumen extending from the first opening to the second opening, wherein the elongate body may have a first location having a first outer diameter configured to be spaced from an inner wall of the valve well and a second location having a second outer diameter configured to engage the inner wall of the valve well and wherein the second location of the elongate body may extend circumferentially around the elongate body.

Alternatively or additionally to any of the examples above, a majority of a length of the elongate body may have the first outer diameter.

Alternatively or additionally to any of the examples above, a wall thickness of the elongate body at the second location may be at least is at least 1.50 mm.

Alternatively or additionally to any of the examples above, the single material may have a durometer in a range of 50 Shore A to 75 Shore A.

Alternatively or additionally to any of the examples above, the elongate body may have a third location and a fourth location longitudinally spaced from the third location, the third location and the fourth location having a third outer diameter, the third outer diameter may be less than the first outer diameter, and the second location may be between the third location and the fourth location.

Alternatively or additionally to any of the examples above, the elongate body may comprise one or more locations with a third outer diameter that is less than the first outer diameter, a first region including a portion having the second outer diameter located between two locations each having the third outer diameter, and a second region including a portion having the second outer diameter located between two locations each having the third outer diameter.

Alternatively or additionally to any of the examples above, the first region may be proximal of the first opening and the second region is distal of the first opening.

Alternatively or additionally to any of the examples above, the elongate body may comprise a third region including a portion having the second outer diameter located between two locations each having the third outer diameter, and the first region may be proximal of the first opening, the second region may be distal of the first opening, and the third region may be distal of the second region.

In another example, a suction valve stem for use in an endoscope having a suction valve well may include an elongate body, a first opening in the elongate body, a second opening in the elongate body, a lumen extending from the first opening to the second opening, and a seal extending circumferentially around the elongate body at a location proximal of the first opening, and wherein the elongate body and the seal may be formed from a same polymer material having a durometer in a range of 50 Shore A to 75 Shore A.

Alternatively or additionally to any of the examples above, the suction valve stem may further include a first recess extending circumferentially around the elongate body, and a second recess extending circumferentially around the elongate body, and wherein the seal may extend from the first recess to the second recess.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of components of an illustrative endoscope system;

FIG. 3 depicts a schematic perspective view of an illustrative valve stem;

FIG. 4 depicts a schematic cross-section view of the illustrative valve stem depicted in FIG. 3;

FIG. 5 depicts a schematic cross-section view of the illustrative valve stem depicted in FIG. 3, where the valve stem is in a valve well;

FIG. 6 depicts a schematic perspective view of an illustrative valve stem;

FIG. 7 depicts a schematic cross-section view of the illustrative valve stem depicted in FIG. 6; and

FIGS. 8A and 8B depict schematic cross-section views of magnified portions of the valve stem depicted in FIG. 6 and positioned within a valve well.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

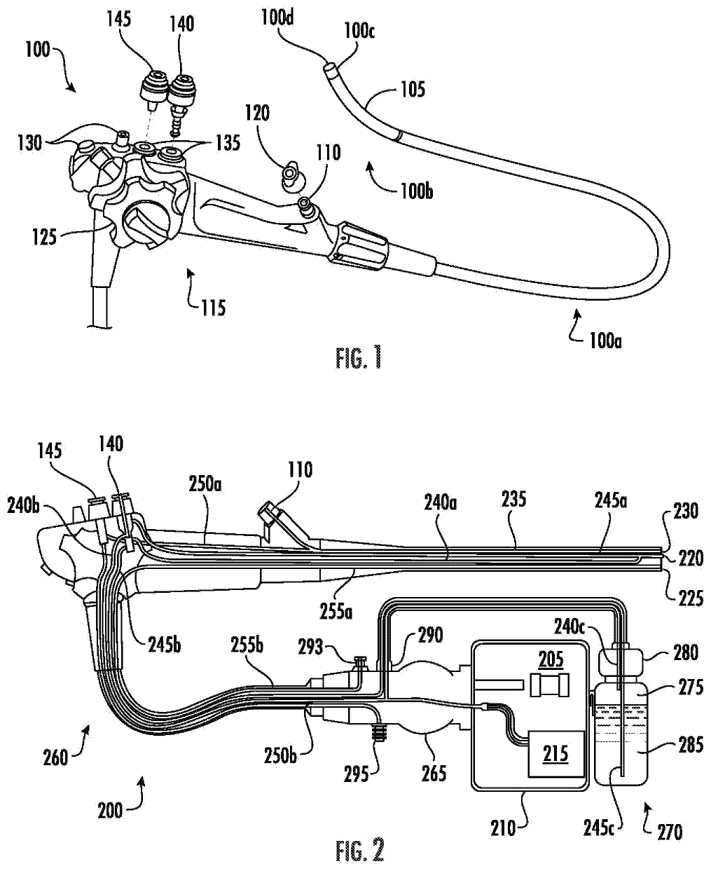

With reference to FIG. 1, an illustrative endoscope 100 is depicted and FIG. 2 depicts an illustrative endoscope system 200. The endoscope 100 may include an elongated tube or shaft 100a that is configured to be inserted into a subject (e.g., a patient).

A light source 205 of the endoscope system 200 may feed illumination light to a distal portion 100b of the endoscope 100. The distal portion 100b of the endoscope 100 may house an imager (e.g., CCD or CMOS imager) (not shown). The light source 205 (e.g., lamp) may be located in a video processing unit 210 that processes signals input from the imager and outputs processed video signals to a video monitor (not shown) for viewing. The video processing unit 210 may also serve as a component of an air/water feed circuit by housing a pressurizing pump 215, such as an air feed pump, in the unit 210.

The endoscope shaft 100a may include a distal tip 100c (e.g., a distal tip unit) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the flexible bending portion 105 (e.g., one knob controls up-down steering and another knob control for left-right steering). A plurality of video switches 130 for remotely operating the video processing unit 210 may be arranged on a proximal end side of the handle 115.

The handle 115 may be provided with dual valve locations 135. One of the valve locations 135 may have or receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220, as depicted in FIG. 2.

The other valve location 135 may have or receive a suction valve 145 for operating a suction operation. A suction supply line 250a may run distally from the suction valve 145 along the shaft 100a to a junction point in fluid communication with the working channel 235 of the endoscope 100.

The operating handle 115 may be electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle and/or other suitable reservoir or container) may be fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, may pass through the top 280 of the reservoir 270 to the same detachable connection 290 as the gas supply tubing 240c on the connector portion 265. In other embodiments, the connections may be separate and/or separated from each other. The connector portion 265 may also have a detachable irrigation connection 293 for irrigation supply tubing (not shown) running from a source of irrigation water (not shown) to the irrigation feed line 255b in the umbilical 260. In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve 140 in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

Referring to FIG. 2, an illustrative operation of an endoscopic system 200, including an endoscope such as endoscope 100 above, is explained. Air from the air pump 215 in the video processing unit 210 may flow through the connection portion 265 and branch to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user's finger on the valve, air is allowed to flow out of the valve 140 to atmosphere. In a first position, the user's finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve 140, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. In some embodiments, outflow check valves or other one-way valve configurations (not shown) may be placed in the path of the lens wash supply tubing to help prevent water from back-flowing into the reservoir 270 after the water has passed the valve.

A relatively higher flow rate compared to lens wash is typically required for irrigation water, since a primary use is to clear the treatment area in the patient of debris that obstructs the user's field of view. Irrigation is typically achieved with the use of a pump (e.g., peristaltic pump), as described. In configurations with an independent water source for irrigation, tubing placed in the bottom of a water source may be passed through the top of the water source and threaded through the head on the upstream side of the pump. Tubing on the downstream side of the pump 255c is connected to the irrigation feed line 255b in the umbilical 260 and the irrigation supply line 255a of endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical 260, and down the irrigation supply line in the shaft 100a of the endoscope 100 to the distal tip 100c. In order to equalize the pressure in the water source as water is pumped out of the irrigation supply tubing, an air vent (not shown) may be included in the top 280 of the water reservoir 270. The vent allows atmospheric air into the water source preventing negative pressure build-up in the water source, which could create a vacuum that suctions undesired matter from the patient back through the endoscope toward the water source. In some configurations, outflow check valves or other one-way valve configurations (not shown), similar to the lens wash tubing 245c, may be placed in the path of the irrigation supply tubing to help prevent back-flow into the reservoir after water has passed the valve.

The suction valve 145 may be configured to allow or prevent suction and/or a suction effect in the working channel 235. When the suction valve 145 is in a valve closed position (e.g., a first configuration), a suction fluid flow through the working channel 235 may be blocked by the suction valve 145. When suction is desired in the working channel 235, an operator or user may actuate the suction valve 145 (e.g., by depressing a button on the valve and/or actuating the suction valve 145 in one or more other suitable manners) in order to bring the suction valve 145 to a valve open position (e.g., a second configuration). When the suction valve 145 is in the valve opened position, a flow channel inside the suction valve may connect the working channel 235 to the suction device coupled to suction connection 295 and the suction device may create a negative pressure that draws fluid into and out of the working channel 235 through an outlet provided in the suction valve. When the operator or user releases the suction valve 145, the valve 145 may return to its valve closed position and reduce or block a suction fluid flow from the working channel 235.

In some cases, suction valves 145 may rely on a path of least resistance to direct a suction fluid flow through the endoscope system 200. In some cases, when a suction pump is turned on for a procedure, the pump remains on for an entirety of the procedure and continually pulls air from the flexible umbilical 260, which in turn draws fluid from the line side of the endoscope 100 that runs up the umbilical 260 and connects to a port at the suction valve 145. When the suction valve 145 is in a first position and/or configuration (e.g., a closed position) the suction force or negative pressure from the suction pump is blocked from the working channel 235 and may pull fluid from atmosphere through the suction valve 145. When the suction valve 145 is actuated to a second position and/or configuration (e.g., an opened position) (e.g., when the button or cap associated with the suction valve 145 is depressed and/or actuated in one or more other suitable manners), the opening from atmosphere through the suction valve 145 to the suction pump may be effectively closed or blocked by the suction valve 145 and a fluid path between working channel 235 and the suction pump through the suction valve 145 may be opened. Thus, fluid moving to the suction pump may follow a path of least resistance, where the path may change depending on whether the suction valve 145 is in a first position (e.g., a closed position) or a second position (e.g., an opened position)

In some cases, valve stems of the gas/water valve 140 and/or the suction valve 145 may be configured to have a close fit with a valve well configured to receive the valve stem in the endoscope 100. In such gas/water valves 140 and suction valves 145, when the valve stem is in a first position (e.g., a closed position) the close fit blocks a flow path or increases a resistance to flow between the working channel 235 and a fluid line (e.g., the gas feed line 240b, lens wash feed line 245b, suction feed line 250b) of the endoscope system 200. Similarly, when the valve stem is in a second position, the close fit opens a flow path and reduces a resistance to flow between the working channel 235 and the fluid lines.

The lens wash valve 140 and/or the suction valve 145 configured to block flow using close fits between the valve stem and valve well require valves stems that are precisely manufactured. The precision required to produce suction valves with close fits has required expensive materials (e.g., metals, etc.), highly precise machinery, and is time consuming to achieve.

Additionally, valves with close fit valve stems and valve wells are manufactured to have at least some clearance to allow the valve stem to adjust positions within the valve well. This clearance, may result in leakage during use, which may lead to at least two issues noticeable by a physician.

The first issue is when the valve is in a position intended to block flow from the working channel 235, there is still some flow passing through the working channel 235. The smaller the clearance between the valve stem and the valve well, the less unwanted flow through the working channel 235 that occurs and the larger the clearance, the more unwanted flow through the working channel 235, however, clearance is needed to facilitate movement of the valve stem within the valve well. When flow is actively moving through the working channel 235 in such configurations of the valve, users may perceive the suction as “poor insufflation” and/or experience leakage at the distal end of the endoscope shaft 100a, even when the valve is in a position intended to block a flow from the working channel 235.

The second issue is when a valve stem of the valve is in a position within a valve well to facilitate a flow between the working channel 235 and fluid lines, the desired flow may be insufficient or weaker than desired or anticipated due to fluid escaping through the clearances from its intended path. In one example, when a valve stem of the suction valve 145 is configured to have a close-fit with a valve well and is in a position within the valve well to facilitate a suction flow between the working channel 235 and the suction pump through the suction valve 145, the flow from atmosphere to the suction pump may not be completely blocked. Any such leaking from atmosphere may reduce a pressure differential between the suction valve 145 and the distal end of the working channel 235, which may lead to a reduced suction force or negative pressure, reduced flow rates, and aerated flow through the fluid path to the suction pump.

Valves configured to operate with close-fit valve stems and valve wells may work well enough when intended for re-use in multiple procedures, as a price point for such valves can be high enough to justify manufacturing the valves from materials and with the necessary precision that can achieve and maintain desired tolerances over the life of the reusable valves. However, a price point of a single use valve may not allow for use of the necessary materials, tools, and/or the precise manufacturing required to achieve and/or maintain tolerances over the life of single-use suction valves.

In some cases, compliant seals may be applied to a central shaft to form a valve stem that is configured to seal off flow paths through a valve in an endoscope. The compliant seals may be applied to the central shaft as discrete components and/or may be applied to the central shaft through insert-molding or over-molding onto the central shaft. However, maintaining quality when assembling discrete seals on the central shafts can be challenging due to the high production volumes and/or other factors. Although applying seals to a central shaft via insert-molding or over-molding may be more efficient than assembling discrete seals on central shafts, insert-molding and over-molding may require special equipment to apply the seals to the central shaft.

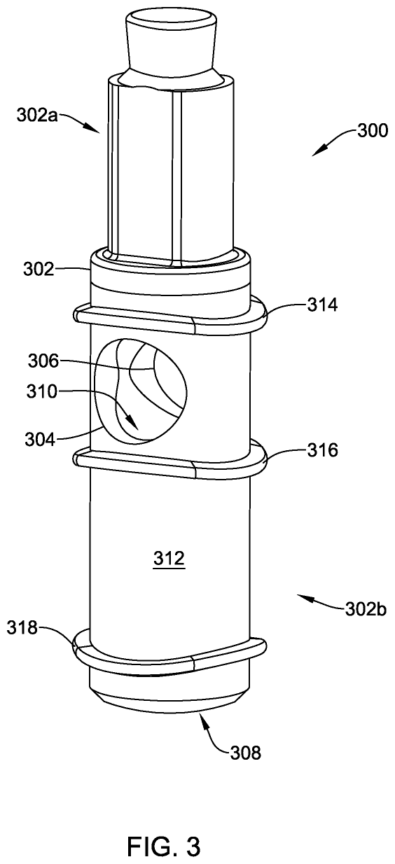

The valve configurations for endoscopes 100 and/or other suitable scopes discussed herein address the above-noted concerns with existing valves and are configured to mitigate and/or eliminate leakage across the valves. The valve configurations discussed with respect to FIGS. 3-8B may include valve stems that are formed integral with one or more seals or other seal features. Such valve stems may be formed from a single material on a standard injection molding machine and/or using one or more other suitable processes.

FIG. 3 depicts a perspective view of an illustrative valve stem 300 for use in an endoscope 100 (e.g., a medical device assembly). Although the valve stem 300 depicted in FIG. 3 may be configured for use in a suction valve 145, the valve stem 300 may be configured for use in a gas/water valve 140 and/or other suitable valves including features and/or properties similar to those of suction valves 145 and/or gas/water valves 140, but with seals and/or openings at different locations and configured for a particular purpose (e.g., where the seals and/or openings may be configured and/or positioned to adjustably align with openings of a valve well).

The valve stem 300 may have any suitable configuration configured to adjust positions within a valve well and/or adjustably fluidly couple the working channel 235 with fluid lines of the endoscope system 200. In one example, the valve stem 300 may be elongated and may include one or more openings and one or more lumens extending between the one or more openings.

The valve stem 300 depicted in FIG. 3 may include an elongate body 302. The elongate body 302 may include a first portion 302a and a second portion 302b. The first portion 302a of the elongate body 302 may be an engagement portion configured to be actuated to adjust a position of the valve stem 300 within a valve well. In some cases, the first portion 302a may be configured to couple with a button or cap for the valve that a user may interact with to adjust a position of the valve stem 300 within the valve well, but this is not required. Example suitable coupling mechanisms for connecting the first portion 302a with a button or cap may include, but are not limited to, adhesives, a threaded connection, a luer lock connection, a snap connection, a ball-detent connector, a friction fit, and/or additional or alternative coupling mechanisms. The first portion 302a of the elongate body 302 may also include one or more “keying” features that are configured to engage keying features of a valve well to enable orientation of the valve stem 300 within the valve well. The second portion 302b of the elongate body 302 may be configured to be positioned within the valve well and may include features for facilitating fluid flow between the working channel 235 and fluid lines of the endoscope system 200 and/or blocking fluid flow between the working channel 235 and the fluid lines.

The valve stem 300 may include one or more openings in the elongate body 302. In one example, as depicted in FIG. 3, the valve stem 300 may include a first opening 304 in the elongate body 302, a second opening 306 in the elongate body 302, and a third opening 308 in the elongate body 302, but other suitable configurations are contemplated. In some cases, one or more of the openings in the valve stem 300 may be a radial or side opening and one or more openings may be axial or end openings. In the example depicted in FIG. 3, the first opening 304 and the second opening 306 may be radial openings located at a same, first axial location along a longitudinal axis of the elongate body 302 and the third opening 308 may be an axial opening located at a second axial location along the longitudinal axis of the elongate body 302 that is distal of the first axial location.

The valve stem 300 may include one or more lumens extending between the one or more openings of the valve stem 300. As depicted in FIG. 3, a lumen 310 may extend between the third opening 308 and the first opening 304 and the second opening 306. Other suitable configurations of the lumen 310 and/or openings 304, 306, 308 that facilitate adjusting flow paths across a valve based on a position of the valve stem 300 relative to a valve well are contemplated.

The valve stem 300 may include one or more seals that are configured to translate with the valve stem 300 within a valve well. The seals of the valve stem 300 may be configured about the elongate body 302 in any suitable manner for interacting with (e.g., contacting) and/or maintaining contact with an inner wall or walls of a valve well in which the valve stem 300 is configured to adjust positions to create a barrier to fluid flow. Illustratively, the seals may extend entirely or at least partially circumferentially around the elongate body 302 of the valve stem 300 and may extend radially outward from an outer surface 312 of the elongate body 302. As depicted in FIG. 3, the valve stem 300 may include a first seal 314, a second seal 316, and a third seal 318, where the first seal 314 may be located proximal of the first opening 304 and the second opening 306, the second seal 316 may be located distal of the first opening 304 and the second opening 306, and the third seal 318 may be located distal of the second seal. Other configurations of the seals of the valve stem 300 are contemplated.

The valve stem 300 utilizing one or more seals may be configured to have increased clearance within a valve well when compared to traditional valve stems configured to create a friction fit with an inner wall or surface of the inner wall of the valve well, and particularly when compared to reusable valve stems. For example, traditional valve stems may be configured to have clearance within a valve well of less than about 0.002 inches, such as a clearance of about 0.002 to about 0.0005 inches, where the elongate body 302 of the valve stem 300 of the present disclosure may have a clearance of at least about 0.005 inches circumferentially between the outer surface 312 in between seals and an inner wall of a valve well. In one example, the elongate body 302 of the valve stem 300 may be sized to (e.g., configured to) have a clearance of about 0.005 inches to about 0.010 inches between the outer surface 312 and the inner wall of a valve well. The ability to form valve stems 300 to have larger clearances within valve wells relative to traditional valve configurations facilitates using less precise manufacturing techniques and/or materials with more variable tolerances to create the valve stem 300 than has traditionally been used for suction valves of endoscopes. Other suitable clearances and/or dimensions are contemplated.

In additional and/or alternative configurations, discrete seals may be omitted from the valve stem 300. In such cases, the outer surface 312 of the elongate body 302 may have a diameter configured to allow the outer surface 312 to engage an inner wall of a valve well in which the valve stem 300 is positioned. When so configured, the outer surface 312 of the elongate body 302 may act as a seal, such that axial adjustment of the valve stem 300 within the valve well may adjust fluid flows through the valve. In some cases, utilizing discrete seals on the valve stem 300 (e.g., as depicted in FIG. 3) may reduce drag as the valve stem 300 translates within the valve well relative to an amount of drag produced by a configuration of the valve stem 300 in which the outer surface 312 engages the inner wall of the valve well as a seal.

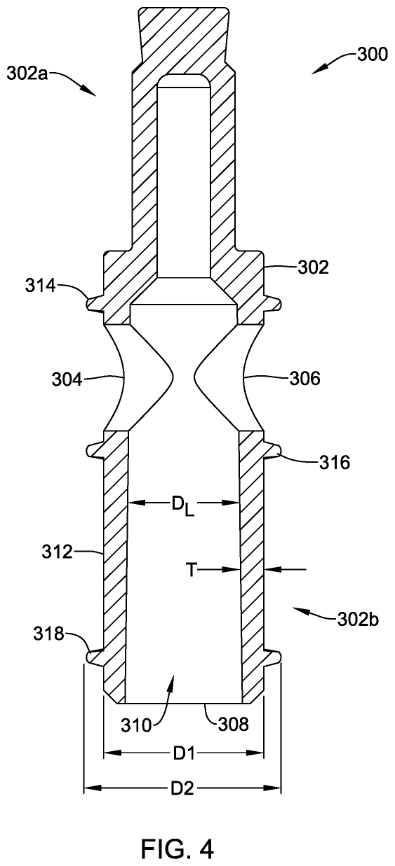

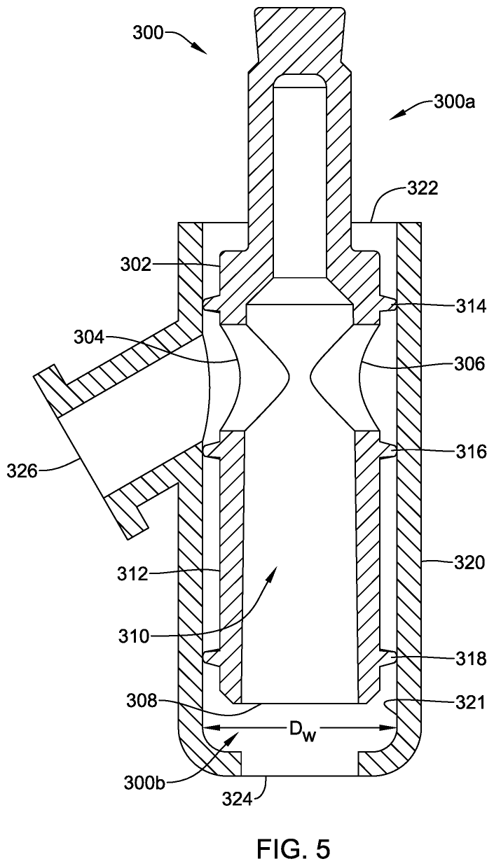

FIG. 4 depicts a cross-sectional view of the valve stem 300 depicted in FIG. 3. FIG. 5 is a schematic cross-section view of the valve stem 300 depicted in FIG. 4 and at least partially positioned in a valve well 320, which is also shown in cross section.

The valve well 320 may have an inner wall 321 with an inner diameter D_{W} and one or more openings. In one example, the valve well 320 may have a proximal opening 322 configured to allow for translation of the valve stem 300 within the valve well 320, a distal opening 324 configured to couple with a first tubing of the endoscope system 200, and an intermediate opening 326 configured to couple with a second tubing of the endoscope system 200.

The inner diameter D_{W} of an inner surface or wall 321 of the valve well 320 may be any suitable size. In one example, the inner diameter D_{W} of the inner wall 321 may be as size in a range of 5.50 millimeters (mm)-8.50 mm, but this is not required and other suitable sizes for the inner diameter D_{W} are contemplated.

As depicted in FIG. 4, the valve stem 300 may be unitarily formed from a single material such that the elongate body 302 and the seals (e.g., the first seal 314, the second seal 316, and the third seal 318) may be monolithic and/or monolithically formed. When monolithically formed, the elongate body 302 may include and/or define the seals, but this is not required.

The single material may be formed from one or more base materials. When the single material is formed from a two or more base materials (e.g., two or more polymers, etc.), the two or more base materials are mixed together to form the single material such that all or substantially all portions of the valve stem formed from the single material have a same or similar material composition.

The entirety or at least a portion of (e.g., the second portion 302b) the elongate body 302 may be formed from a same, single material as the first seal 314, the second seal 316, and the third seal 318, such that the elongate body 302, the first seal 314, the second seal 316, and the third seal 218 may be monolithic. In some cases, the valve stem 300 may be formed from a single-shot injection molding process, but other suitable processes for forming the elongate body 302 are contemplated.

The valve stem 300 and/or components thereof formed from the single material may be formed to have any suitable material hardness. In some cases, the valve stem 300 and/or components thereof may be formed from any suitable material having a hardness in a range of durometers of about 20-80 shore A, about 30-60 shore A, about 50-75 shore A, and/or other suitable values within one or more other suitable ranges of durometer, but could be softer or firmer depending on the geometry used for valve stem 300 and/or components thereof and an inner wall of a valve well.

The valve stem 300 and/or components thereof formed from the single material may be formed from any suitable material. Example suitable materials for the single material include, but are not limited to, a polymer, thermoplastic elastomers (TPE), thermoplastic polyurethane (TPU), liquid silicone rubber (LSR), and/or other suitable materials. In one example, the single material may be a TPE, which is a blend of thermoplastic and elastomeric polymers that exhibits qualities of both polymer types. In a further example, the single material may be a TPE having a durometer in a range of 50 Shore A to 75 Shore A. Using a TPE as the single material to form the valve stem 300 may facilitate manufacturing the valve stem 300 as TPEs are able to be processed and molded in standard molding machines (e.g., single-shot injection-molding machines, etc.), whereas other materials (e.g., liquid silicon rubbers, etc.) may require specialized molding equipment to form the valve stem 300.

In some cases, one or more portions of the valve stem 300 may be configured to have various predetermined diameters and thickness of the elongate body 302, as depicted in FIGS. 4 and 5. In some cases, the diameters of the valve stem 300 may depend on and/or may be configured based on an inner diameter and/or configuration of a valve well in which the valve stem 300 is intended to translate and/or may be based on one or more other suitable factors.

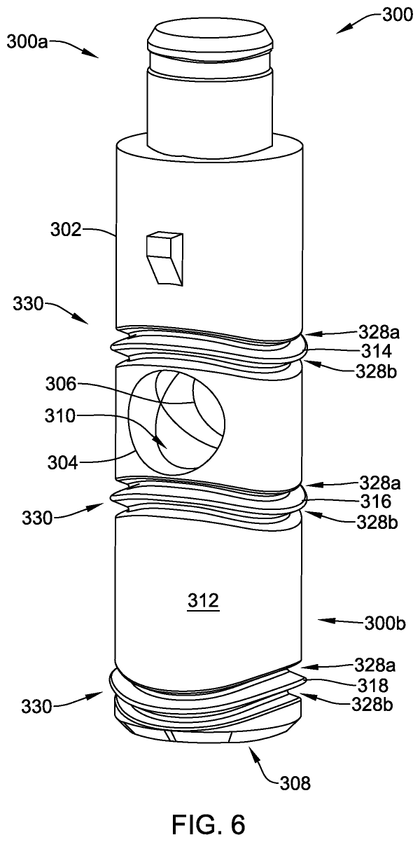

In some examples, the second portion 302b of the elongate body 302 may include a diameter D_{L} of the lumen 310 and may have one or more areas with a first or primary diameter D1 and one or more areas with a second or secondary diameter D2, where the secondary diameter D2 is greater than the first diameter D1. In one example, the primary diameter D1 may be a diameter of an outer surface 312 of the elongate body 302 (e.g., at an axial location between the first seal 314 and the second seal 316 and/or at one or more other suitable axial locations) and the secondary diameter D2 may be a diameter of the radially outer-most point of the first, second, and third seals 314, 316, and 318 (e.g., the valve stem 300 may have three regions with the secondary diameter D2, with at least one seal region 330 proximal of the of the first opening 304 and at least one region 330 distal of the first opening 304 (see e.g., the seal regions 330 labeled in FIG. 6)), where the primary diameter D1 and the secondary diameter D2 may extend entirely or at least partially circumferentially around the elongate body 302 and/or other suitable portion of the valve stem 300. Although not required, a majority of a length of the elongate body 302 may have the primary outer diameter D1.

The diameter D_{L} of the lumen 310 may be any suitable size. In some cases, the diameter D_{L} of the lumen 310 should not be a choke point in a fluid path and as such, may be at least a size of a diameter of a largest lumen of the working channel 235 of the endoscope 100. In one example, lumen 310 may have a diameter D_{L} of 3.80 mm or larger, but this is not required and other suitable diameters are contemplated.

The second portion 302b of the elongate body 302 may have a wall thickness T of any suitable size. The size of the wall thickness may be determined, at least in part, on a type of material used to form the elongate body 302 and/or one or more other suitable factors. In some cases, the wall thickness T at the second portion 302b of the elongate body 302 may be sized to accommodate the diameter D_{L} of the lumen 310, while allowing the second portion 302b of the elongate body 302 to fit within and translate within the valve well 320 without buckling. In some examples, the wall thickness T may be at least 1.00 mm in a range of 1.00 mm to 2.00 mm, is at least 1.50 mm, is in a range of 1.50 mm to 2.00 mm, and/or is one or more other suitable sizes.

The primary diameter D1 of the valve stem 300 or elongate body 302 may be any suitable distance. In some cases, the primary diameter D1 may, at least in part, be determined based on a material type used to form the elongate body 302 and be as large as possible relative to a diameter of an inner wall 321 of the valve well 320 so as to maintain column strength of the valve stem 300 and prevent buckling of the elongate body 302, while allowing for movement of the valve stem 300 in the valve well 320. When the valve stem 300 includes one or more discrete seals (e.g., the first, second, and third seals 314, 316, 318), the primary diameter D1 may be sized to be spaced from the inner wall 321 of the valve well 320 and provide space for the seals to bend or flex when engaging the inner wall 321. In some cases, the primary diameter D1 may have a size in a range between D_{L}+2*Tₘᵢₙ and D_{w}+0.30 mm, where D_{L} is the diameter of the lumen 310, Tₘᵢₙ is a minimum acceptable wall thickness T, and D_{w} is the inner diameter of the inner wall 321 of the valve well 320. For example, the primary diameter D1 may be in a range of about 5.80 millimeters (mm) to about 8.80 mm. Other suitable sizes and/or configurations of the primary diameter D1 of the valve stem 300 are contemplated.

The secondary diameter D2 of the valve stem 300 may be any suitable distance. In some cases, the secondary diameter D2 may be greater than the primary diameter D1 and/or the diameter D_{w} of the inner wall 321 of the valve well 320, such that the valve stem 300 (e.g., the elongate body 302 of the valve stem 300) may include a location having the secondary diameter D2 configured to engage or fluidly seal against the inner wall 321 of the valve well 320. In one example, the secondary diameter D2 may be configured to be 0.10 mm to 0.30 mm greater than the diameter of the inner wall 321 of the valve well 320 to ensure the seals maintain contact with the inner wall 321 and sufficiently flex or bend in response to engagement with the inner wall 321 of the valve well 320 as the valve stem 300 translates within the valve well 320. In another example, when the diameter of the inner wall 321 of the valve well 320 is about 7.00 mm, the primary diameter may be about 6.80 mm, and the secondary diameter D2 may be in a range of 7.10 mm to 7.30 mm. Other suitable configurations of the secondary diameter D2 are contemplated.

FIGS. 6-8B depict schematic views of a configuration of the valve stem 300 having one or more recesses 328a, 328b proximate the seals (e.g., the first seal 314, the second seal 316, the third seal 318, etc.) FIG. 6 depicts a schematic perspective of the valve stem 300 with a first recess 328a and a second recess 328b proximate the first, second, and third seals 314, 318, 316, where the first recesses 328a and the second recesses 328b extend circumferentially around the elongate body 302.

As depicted in FIG. 6, each seal of the valve stem 300 combined with the adjacent or proximate recesses 328a, 328b may form a seal region 330. Utilization of the recesses 328a, 328b may facilitate axial movement of the valve stem 300 with less force than if the recesses 328a, 328b were not included (e.g., omitted) because recesses 328a, 328b may allow for deflection the sealing surfaces (e.g., the seals 314, 316, 318) and the deflection of the sealing surfaces reduces drag on the inner wall 321 of the valve well 320 as the valve stem 300 translates within the valve well 320.

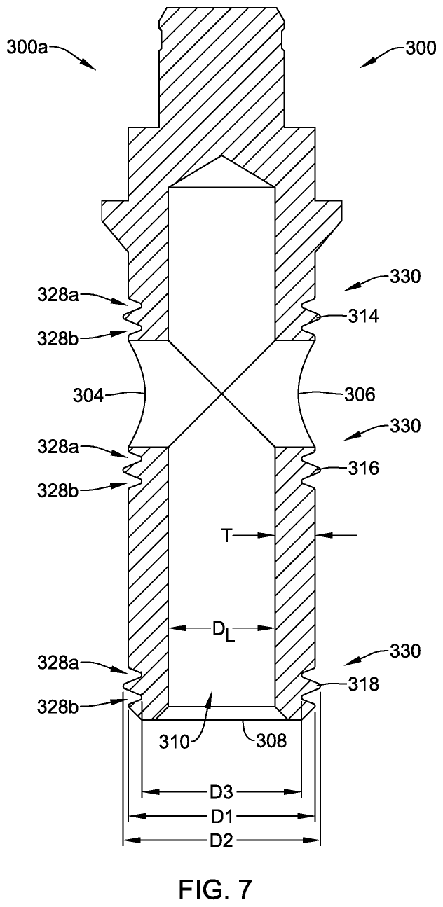

FIG. 7 is a schematic cross-section view of the configuration of the valve stem 300 depicted in FIG. 6 with three seal regions 330 having recesses 328a, 328b, where a first seal region 330 is proximal of the first opening 304, the second seal region 330 is distal of the first opening 304, and the third seal region 330 may be distal of the second seal region 330. As depicted in FIG. 7, a seal (e.g., the first seal 314, the second seal 316, and/or the third seal 318) of a seal region 330 may extend from the first recess 328a to the second recess 328b, but this is not required.

The recesses 328a, 328b may have any suitable size and/or shape. In some examples, the recess 328a, 328b may have cross-sections having a u-shape, a v-shape, a step-shape, and/or other suitable shape.

A third diameter D3 of the valve stem 300 or elongate body 302 may be located at the recesses 328a, 328b and may be any suitable distance sufficient to allow the seals 314, 316, 318 to flex or bend to facilitate translation of the valve stem 300 within a valve well. The third diameter D3 may be at an axial location of the recess 328a, 328b that has the smallest diameter, at an axial location of the recess 328a, 328b at which two surfaces of the recess 328a, 328b merge, or at another suitable location. In one example, the third diameter D3 of the valve stem 300 at the recesses 328a, 328b may be in a range of 1.00 mm to 3.00 mm less than the primary diameter D1. In one example configuration, the primary diameter D1 of the valve stem 300 may be about 6.90 mm, the secondary diameter D2 of the valve stem 300 may be about 7.10 mm, the minimum diameter D3 of the valve stem 300 may be about 6.80 mm, and the diameter of the inner wall 321 of the valve well 320 may be about 7.00 mm.

The recess 328a, 328b may have any suitable width and/or angle from the outer surface 312 of the valve stem 300 to the minimum diameter D3 of the valve stem 300 at the recesses 328a, 328b that facilitates bending and flexing of the seals in response to engagement with the inner wall 321 of the valve well 320.

As discussed, the recesses 328a, 328b may facilitate translation of the valve stem 300 within a valve well by providing space proximate the seals 314, 316, 318 for the seals 314, 316, 318 to move in response to engagement of the seals 314, 316, 318 with the inner wall 321 of the valve well 320. Further, as a result of the recesses 328a, 328b being a reduced diameter location that facilitates movement of the seals 314, 316, 318 as the valve stem 300 translates within a valve well, the primary diameter D1 and the wall thickness T at the primary diameter D1 may be increased to a size closer to a size of the secondary diameter D2 than would be acceptable without the recesses 328a, 328b, which improves an overall column strength of the valve stem 300 and facilitates using TPEs and/or other suitable materials for the elongate body 302.

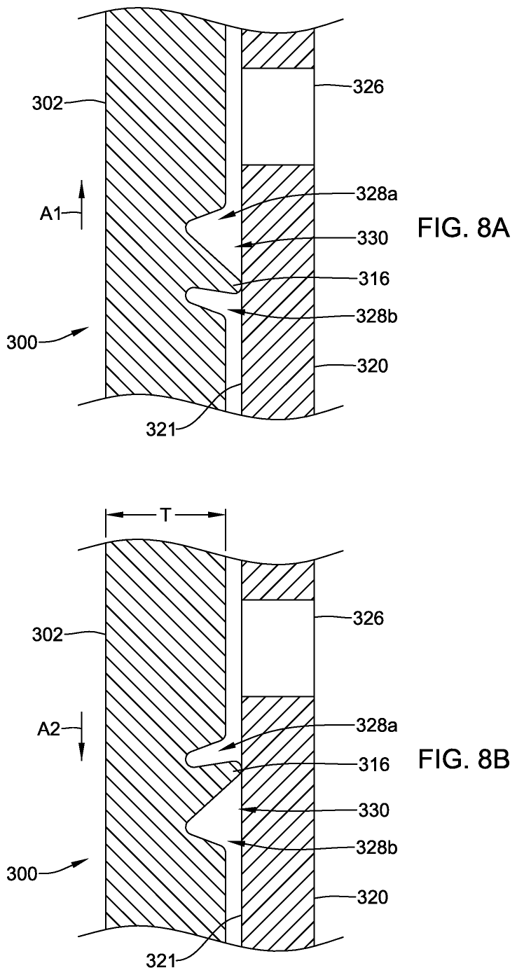

FIGS. 8A and 8B depict the magnified portions of schematic cross-section views of the valve stem 300 depicted in FIG. 6 at a seal region 330 thereof within a valve well 320. FIG. 8A depicts the valve stem 300 translating in a proximal direction of arrow A1 relative to the valve well 320, with the second seal 316 (e.g., a seal located distal of the second opening 306 or located at one or more other suitable locations) flexing or bending into the recess 328b in response to engagement with the inner wall 321 of the valve well 320. FIG. 8B depicts the valve stem 300 translating in a distal direction of arrow A2 relative to the valve well 320, with the second seal 316 flexing or bending into the first recess 328a in response to engagement with the inner wall 321 of the valve well 320.

As shown in FIGS. 8A and 8B, the recesses 328a, 328b may provide space for the seals of the valve stem 300 to flex or bend as the valve stem 300 translates within the valve well 320. As such, the space created by the recesses 328a, 328b may facilitate forming a thicker wall thickness T and/or a greater primary diameter D1 of the elongate body 302 of the valve stem 300 relative to the wall thickness T and/or primary diameter D1 of the valve stem 300 depicted in FIGS. 4 and 5, assuming the respective valve stems 300 have a same lumen diameter D_{L} and are configured for use in valve wells having the same inner diameter, because the primary diameter D1 no longer needs to be sized to facilitate flexing or bending of the seals.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A valve stem for a medical device, the valve stem comprising:
an elongate body;
a first opening in the elongate body;
a second opening in the elongate body;
a lumen extending from the first opening to the second opening;
a first seal extending circumferentially around the elongate body at a location proximal of the first opening; and
a second seal extending circumferentially around the elongate body at a location distal of the first opening;
wherein the elongate body, the first seal, and the second seal are formed from a single material.

2. The valve stem of claim 1, wherein the elongate body, the first seal, and the second seal are monolithic.

3. The valve stem of claim 1, further comprising:
a third seal extending circumferentially around the elongate body at a location distal of the second seal,
wherein the third seal is formed from the single material used to form the elongate body, the first seal, and the second seal.

4. The valve stem of claim 1, further comprising:
a first recess extending circumferentially around the elongate body at a first axial location; and
a second recess extending circumferentially around the elongate body at a second axial location spaced from the first axial location,
wherein the first seal extends from the first recess to the second recess.

5. The valve stem of claim 1, wherein the single material is a thermoplastic elastomer.

6. The valve stem of claim 1, wherein a wall thickness of the elongate body between the lumen and an outer surface of the elongate body at an axial location between the first seal and the second seal is in a range of 1.00 millimeters (mm) and 2.00 mm.

7. The valve stem of claim 1, wherein a wall thickness of the elongate body between the lumen and an outer surface of the elongate body at an axial location between the first seal and the second seal is at least 1.50 mm.

8. The valve stem of claim 1, wherein the single material has a durometer in a range of 50 Shore A to 75 Shore A.

9. The valve stem of claim 1, wherein the first opening is a radial opening and the second opening is an axial opening in a distal end of the elongate body.

10. The valve stem of claim 1, wherein an engagement portion extends proximally from the elongate body.

11. A valve stem for a medical device, the valve stem comprises:
an elongate body formed from a single material;
a first opening in the elongate body;
a second opening in the elongate body; and
a lumen extending from the first opening to the second opening;
wherein the elongate body has a first location having a first outer diameter configured to be spaced from an inner wall of the valve well and a second location having a second outer diameter configured to engage the inner wall of the valve well; and
wherein the second location of the elongate body extends circumferentially around the elongate body.

12. The valve stem of claim 11, wherein a majority of a length of the elongate body has the first outer diameter.

13. The valve stem of claim 11, wherein a wall thickness of the elongate body at the second location is at least is at least 1.50 mm.

14. The valve stem of claim 11, wherein the single material has a durometer in a range of 50 Shore A to 75 Shore A.

15. The valve stem of claim 11, wherein:
the elongate body has a third location and a fourth location longitudinally spaced from the third location, the third location and the fourth location having a third outer diameter,
the third outer diameter is less than the first outer diameter, and
the second location is between the third location and the fourth location.

16. The valve stem of claim 11, wherein the elongate body comprises:
one or more locations with a third outer diameter that is less than the first outer diameter;
a first region including a portion having the second outer diameter located between two locations each having the third outer diameter; and
a second region including a portion having the second outer diameter located between two locations each having the third outer diameter.

17. The valve stem of claim 16, wherein the first region is proximal of the first opening and the second region is distal of the first opening.

18. The valve stem of claim 16, wherein the elongate body comprises:
a third region including a portion having the second outer diameter located between two locations each having the third outer diameter,
wherein the first region is proximal of the first opening, the second region is distal of the first opening, and the third region is distal of the second region.

19. A suction valve stem for use in an endoscope having a suction valve well, the suction valve stem comprising:
an elongate body;
a first opening in the elongate body;
a second opening in the elongate body;
a lumen extending from the first opening to the second opening; and
a seal extending circumferentially around the elongate body at a location proximal of the first opening,
wherein the elongate body and the seal are formed from a same polymer material having a durometer in a range of 50 Shore A to 75 Shore A.

20. The suction valve stem of claim 19, further comprising:
a first recess extending circumferentially around the elongate body; and
a second recess extending circumferentially around the elongate body,
wherein the seal extends from the first recess to the second recess.
